(19) United States
(12) Patent Application Publication
(10) Pub. No.: US 2011/0040236 A1
(43) Pub. Date: Feb. 17, 2011

(54) MICRONEEDLE ROLLER

(75) Inventors: Elliot Isaacs, London (GB); Toby Cobbledick, London (GB)

Correspondence Address: CROSE LAW LLC, 112 NORTHBROOKE TRACE, WOODSTOCK, GA, 30188, US

(73) Assignee: PANGAEA LABORATORIES, LTD., London (GB)

(21) Appl. No.: 12/857,516

(22) Filed: Aug. 16, 2010

(30) Foreign Application Priority Data
Aug. 17, 2009 (GB) GB0914273.8

(51) Int. Cl. A61M 5/00; A61N 7/00; A61N 5/06; A61N 5/067

(52) U.S. Cl. 604/20; 604/173; 604/22

(57) ABSTRACT

The invention provides a microneedle roller comprising a rotatable drum, the drum including a plurality of radially projecting microneedles, wherein the microneedles are provided in substantially linear arrays, each array being carried by a support, and each array being arranged in a substantially axial direction relative to the drum.

### CROSS-REFERENCE TO RELATED APPLICATION

The present non-provisional patent application claims the benefit of priority of foreign Patent Application No. GB0914273.8, which is entitled A MICRONEEDLE ROLLER and which was filed Aug. 17, 2009 which is incorporated in full by reference herein.

### FIELD OF THE INVENTION

The present invention relates to a microneedle roller.

### BACKGROUND OF THE INVENTION

In US2008161735 a microneedle roller is described as being constructed of a plurality of discs mounted on a central axle. Each disc comprises an array of radially oriented microneedles.

### BRIEF SUMMARY OF THE INVENTION

The present invention improves on this construction by providing, in a first aspect, a microneedle roller comprising a handle and a rotatable drum, the drum including a plurality of radially projecting microneedles, the roller further comprising sterilising means in the form of either or both of an ultrasound generator and a UV light for sterilising one or more of the drum, the microneedles, and the surface over which the drum moves in use.

The microneedle roller may comprise a rotatable drum, the drum including a plurality of radially projecting microneedles, wherein the microneedles are provided in substantially linear arrays, each array being carried by a support, and each array being arranged in a substantially axial direction relative to the drum.

The drum may substantially be a right cylinder which may be hollow, however, oblique rollers are also contemplated, as are other forms, such as a barrel-shape with convex or concave sides, or even a sphere.

The supports may be substantially blade-like structures with the microneedles provided along one edge forming a substantially linear array. In this way each support, and thus each array of needles, is arranged substantially parallel with the axis of the drum. This is perpendicular to the arrangement of the known disc roller.

The arrays may be located in axial grooves provided across the radial outer surface of the drum. The supports may be glued into the grooves, or otherwise held therein. Alternatively, the grooves could be replaced by slots which provide openings through the outer radial wall of the drum (assuming the drum is substantially hollow). The arrays of microneedles may then protrude through the slots with their respective supports being held, from inside the drum, or otherwise maintained in position by any relevant mechanical means.

This arrangement has significant benefits compared to the prior art, for instance, a greater density of needles per unit area across the surface of the drum is possible. Another benefit is that the construction is less expensive as there are fewer parts for assembly. In some embodiments the microneedles may have a substantially narrower gauge (diameter) than known prior art rollers due to the strengthening provided by the support. Yet another benefit is that the arrays of microneedles may be arranged around the circumference of a roller of any diameter without the requirement for different sized arrays. Other advantages and/or benefits will become apparent from the following.

Each array of microneedles and its support may be unitary in that they are formed from one piece of material. In this regard the material may be one of a metal, a ceramic, a polymer, a biodegradable polymer, a mineral such as glass, an alloy etc. In one embodiment the support and microneedles are stamped from metal. The microneedles may be hollow and/or transparent to allow for the direction of fluids, and/or thermal energy, and/or electromagnetic radiation onto or away from a surface in use.

In another embodiment, each array of microneedles and its support may be connected together. For instance, this may be by use of adhesive, welding or other mechanical or chemical connection. In this way different materials may be used for the support and the microneedles, however, it is contemplated that they may comprise the same materials.

The support may include an abrasion means for abrading the skin. It is known that abrasion may affect the skin, for instance promoting the release of growth-factors. The abrasion means may be a roughened surface or an abrasive material provided adjacent at least one microneedle. The abrasion means may be provided between adjacent microneedles. The abrasion means may be located on the drum surface.

The surface of the drum may provide abrasion by including a suitable outer surface with or without vibration.

The roller may include adjustment means for varying the radial projection of at least some of the microneedles relative to the drum. This is useful as microneedles of different lengths are required for different purposes. For example, relatively long microneedles are required for clinical therapies such as Platelet Rich Plasma, and relatively shorter microneedles are required to improve or provide transdermal drug penetration. Further, a user may wish to use different length microneedles for different treatment types and/or different treatment areas. A means for adjusting the needle length will mitigate the requirement for several fixed length versions.

The adjustment means may include opposed surfaces, such that one surface may be provided on an adjustment member axially movable relative to, and inside, the drum, and the other surface may be provided on at least one of said supports, wherein said surfaces are substantially parallel with each other and inclined relative to a central axis of the drum. In this way, the two opposing surfaces have oppositely inclined surfaces, such that when they are moved axially relative to one another the radial distance between the support and the central axis of the drum may be varied.

This may be effected by one said surface being provided by the outer face of the adjustment member which is either conical or frusto-conical in shape. The other said surface may be provided by the base of the said support, although other constructional variations are possible.

The adjustment member may be screwable axially into and out of the drum. In this way the adjustment member and the drum may comprise corresponding screw threads. The pitch of the screw threads may vary the controllability of the radial projection adjustment.

The adjustment may also be via non-axial motion, delivered by mechanical means such as an expanding scroll or a rotating spiral. The mechanical means may include a means of resilient bias to bias the microneedles radially inward or outward. The adjustment means may include an hydraulic and/or pneumatic component, or be otherwise based on pressure change. The adjustment means may include an electric motor to act upon a mechanical means.

The roller may further comprise control means for incrementally moving the adjustment means. For instance, the control means may include one of notches or opposing projections provided on the adjustment means and the other provided on the drum. This may form a ratchet-type system where the adjustment means may be moved axially into/out of the drum with a positive “click” being felt and/or heard by an operator/user.

The roller may further comprise indicia for indicating the relative or absolute radial projection of the said at least some of the microneedles relative to the drum. For instance, one of the drum and adjustment means may comprise numerals indicating the length of radial projection in micrometers or millimetres, the other of the drum and adjustment means may comprise an indicator which rotates relative to, and aligns with, the relevant numeral.

The control means and/or the adjustment means may be located remotely from the drum, for instance, from an associated handle. The adjustment means may be controlled by transmission of electronic or light impulse, or by mechanical means such as a system of levers and/or hydraulic and/or pneumatic means.

The radial projection of at least some of the microneedles relative to the outer surface of the drum may be variable between 0 and 5 mm. The radial projection of the microneedles relative to the drum may not be variable, and may range between 0.01 and 5 mm. Individual microneedles, or microneedle arrays, may differ in radial projection to other microneedles or microneedle arrays on the same roller.

The roller may further comprise treatment means for treating one or more of the drum, the microneedles, the roller and the surface over which the roller moves in use. In this regard the term “treating” is used to mean “affecting” in some way. This may be a chemical, electrical, mechanical, or other type of effect. It may be for the purpose of providing medical or cosmetic treatment and/or for improvement of the operation of the roller.

The treatment means may have sterilisation properties. The sterilisation may occur constantly or intermittently in use. Alternatively, or additionally, the treatment means may have skin and/or hair enhancement properties. Alternatively, or additionally, the treatment means may have pain and/or inflammation controlling properties.

The treatment means may comprise an emission or removal of one or more of a liquid, a gas, a sound, or an electro-magnetic radiation. It is also possible that the treatment means comprises temperature adjustment means for varying the temperature of the roller and/or the surface in use. The treatment means may comprise a means of irrigating the microneedles and/or the drum and/or a surface in use.

The treatment means may comprise a brush and/or a comb. This may be useful for providing a scrubbing and/or cleaning effect of either the roller and/or the needles and/or the surface in use. Additionally the treatment means may emanate from and/or be conducted or directed by the brush/comb. The brush and/or comb may be useful in controlling the direction of hair as the roller passes across a surface comprising such hairs, preventing the tangling of hair around the roller, thereby facilitating movement of the roller in multiple directions without impedance or degradation of effectiveness.

The treatment means may comprise a light and/or a laser-emitter. The laser may be a cold-beam laser. The light emitted may lie in the ultraviolet range although other wavelengths of the light spectrum are contemplated, such as in the range 500 to 900 nm. The treatment means may comprise a plurality of sources of laser and/or non-laser light, of ultraviolet and/or another wavelength.

The treatment means may comprise vibration means for imparting a vibration or sound to the roller and/or the surface in use. The vibration may be created by one or more of an electrical off-set motor, a piezo-electric crystal and an ultrasound generator. The ultrasound generator may generate ultrasound in a frequency range of 20 kHz to 200 MHz.

The gas emitted may be ozone. The liquid emitted may be one or more of a hair-growth stimulant, a sterilising fluid, and a pain or inflammation controlling fluid. The electro-magnetic radiation emitted may be gamma, microwave, infra-red and/or beta nuclear radiation. The sound emitted may be ultrasound and/or another frequency sound that may resonate. It may have a frequency lying in the range 20 kHz to 200 MHz.

All of these different physical emissions may be useful in having a beneficial effect on the surface and/or the roller itself. For instance, as at least a partially continuous sterilisation process, and/or a hair growth stimulation process, and/or a pain and/or inflammation controlling process.

The roller may further include a handle relative to which the drum is rotatable. This handle may comprise a roller hood and/or a comb. The treatment means may emanate from the hood and/or comb. For instance the hood and/or comb may act as a light guide for transferring light from a light or laser light source or sources located in the handle and for directing it onto the surface in use, or onto the drum and/or microneedles. Alternatively the source may be located in the hood and/or comb itself. The hood and/or comb may prevent accidental contact with the microneedles by a user.

The vibration and or sound waves may be of an amplitude, wavelength, or resonance, to allow them to be transferred to the roller or surface in use from an emitter in the handle.

In this way, the treatment means may be located in the handle. However, it is also contemplated that the drum comprises the treatment means. It is also possible that the treatment means is located in both or conducted through both or either.

The treatment means may be emitted, conducted or reflected from a brush/comb structure as mentioned above. The structure may also or alternatively comprise of a hood or plate that may project substantially radially around the drum. Any or all of the hood, plate, brush/comb may project from a handle, or a supporting arm for the drum. Alternatively any or all of the hood, plate, brush/comb may project radially from one or more arms of the roller, possibly in the form of a circle or part circle, in order to direct the treatment means axially across the drum and/or the surface in use.

The roller may include one or more treatment means and means for a user to choose or select which one or more of these treatment means are in operation at any one time. The roller may include means for disabling any or all of the treatment means as required.

The handle may have only one arm for supporting the drum. In this way, the arm may support the drum at one axial end-face. The end-face may include an axle or an axle may extend through the drum body. The arm may include means for attachment to the axle such that the drum may rotate relatively freely relative to the handle. In another embodiment, the handle may have two arms for supporting the drum.

The roller may further comprise release means for remotely releasing the drum from a supporting arm. This would be useful in preventing contact with the microneedles after use. This may be important since the microneedles may have some blood residues and may be relatively sharp.

The release means may comprise an axle which extends from a push button located on the arm or opposite end face. This axle may expand and contract radially as required such that with the axle inside the drum body in its expanded state it engages with the drum body to provide support thereto, but in its contracted state is readily removed from inside the drum body. The radial expansion and contraction of the axle may be effected by means of a push button or the like provided on the arm or opposite end face.

The release means may also deform part of the drum and/or axle, and/or attachment means of the drum and/or axle to the handle in order to disengage the drum. The release means may extend or contract the length of the axle to disengage the drum.

Another possibility is that a means of increasing the opening between the two arms is provided. In this way as the arms are moved apart the drum may fall out under the influence of gravity or by shaking the handle.

Any of the above release means may be controlled indirectly or remotely, such as from the handle, via a relevant mechanical means and/or electrically or light conductive means.

In one embodiment, the microneedles and/or the drum may be electrically connected directly or via the microneedle support(s) to at least a portion of the handle. This may allow, for instance, the charge on the microneedles to be adjusted as desired.

The roller may further include biasing means for resiliently biasing the microneedles radially inward or outward relative to the roller. This biasing means may be a resilient member, such as provided by a thermoplastic elastomer (TPE), provided between the support and a portion of the drum.

The biasing means, and other features, may be important as the device will be suitable for use in zero or microgravity.

The roller may include an integral, and therefore portable, power supply. For instance, the roller may have space for receiving one or more power cells such as batteries. Alternatively, or additionally, the roller may include means for recharging a power cell. For instance, the roller may include means for converting kinetic energy, imparted to the roller in use, or by being shaken, to electrical energy.

The roller may include connection means for connecting the roller to an external power source. The power source may be for supplying power to features of the roller directly, or to recharge an integral power supply.

The roller may comprise materials which are suitable for autoclaving in that the materials do not deteriorate and the performance of the roller is not impaired.

The roller or component parts of the roller may be biodegradable to minimise environmental impact of disposal.

The handle may comprise more than one part, the various parts being removably attachable to one another. For instance, the roller may include a head to which the drum is attached. If it is necessary to replace the drum then it is possible that in one embodiment, the whole of the front part of the roller is removed and a new front part including a new roller is attached to the handle.

The connection between adjacent parts of the roller may include articulation and/or resilient means.

The roller may include torque control means for adjusting the torque required to rotate the drum relative to the handle.

The roller may also be used as part of and/or attached to an assembly that may include a handle extension that may be articulated. The assembly may irrigate the roller or a surface in use with a fluid. The assembly may include gripping means for the invention to be held without using hands, and/or allow multiple rollers to be used at one time. The assembly may include a heating or refrigeration source, a source of electromagnetic radiation, an infrasonic, sonic, or ultrasonic emitter, a comb, brush or other device for styling purposes and/or to part the hair for better microneedle treatment, or any combination of these features.

The roller may be used with a topical treatment including any cosmetically active agent (defined as a compound (natural or synthetic) that has a cosmetic or therapeutic effect on the skin, hair or nails including but not limited to lightening agents, darkening agents, anti-acne agents, shine control agents, anti-microbial agents, anti-inflammatory agents, anti-mycotic agents, anti-parasite agents, external analgesic, sunscreens, photo-protectors, antioxidants, keratolytic agents, detergents or surfactants, moisturisers or humectants, nutrients, vitamins, energy-enhancers, growth factors, anti-perspiration agents, astringents, deodorants, hair-removers, firming agents, anti-callous agents and agents for hair, nail and/or skin conditioning.) Of particular interest are curcumin, taurine, plant sterols, pine bark extract, green tea, red tea, white tea, horsetail extract, marine cartilage, caffeine, kieslerde, copper peptides, copper pca, euk-134, copper(II) 3,5-diisopropylsalicylate, minoxidil and other natural or synthetic nitric oxide donators, finasteride, dutasteride, spironolactone, superoxide dismutase (and mimetics), dimethylmethoxy chromanol, catalase mimetics, saw palmetto and other natural and synthetic anti-dihydrotestosterone agents, hydrolysed lupine protein, vitamins c, a, e, b, f, h, k (and derivatives), bacterial filtrates, glucosamine sulphate, or any combination of these.

The roller may be provided as part of a kit that may comprise a sterilising solution, one or more microneedle roller drums, a handle, a rechargeable power source, a replaceable battery, or a cable for mains electricity with or without a transformer, a topical treatment including any cosmetically active agent (defined as a compound (natural or synthetic) that has a cosmetic or therapeutic effect on the skin, hair or nails including but not limited to lightening agents, darkening agents, anti-acne agents, shine control agents, anti-microbial agents, anti-inflammatory agents, anti-mycotic agents, anti-parasite agents, external analgesic, sunscreens, photo-protectors, antioxidants, keratolytic agents, detergents or surfactants, moisturisers or humectants, nutrients, vitamins, energy-enhancers, growth factors, anti-perspiration agents, astringents, deodorants, hair-removers, firming agents, anti-callous agents and agents for hair, nail and/or skin conditioning.) Of particular interest are curcumin, taurine, plant sterols, pine bark extract, green tea, red tea, white tea, horsetail extract, marine cartilage, caffeine, kieslerde, copper peptides, copper pca, euk-134, copper(II) 3,5-diisopropylsalicylate, minoxidil and other natural or synthetic nitric oxide donators, finasteride, dutasteride, spironolactone, superoxide dismutase (and mimetics), dimethylmethoxy chromanol, catalase mimetics, saw palmetto and other natural and synthetic anti-dihydrotestosterone agents, hydrolysed lupine protein, vitamins c, a, e, b, f, h, k (and derivatives), bacterial filtrates, glucosamine sulphate, or any combination of these. The kit may be enclosed completely or partially in a case, cover or container for convenient storage and transport of some or all of the articles in the kit.

The roller may be adaptable or modifiable to allow the use of more than one microneedle roller drum at one time, in parallel, tandem, or any other arrangement. This may be achieved by means of expanding the arms of the roller handle, or using a longer axle on an embodiment utilising only one supporting arm. This may additionally or alternatively be achieved by the roller handle being comprised of additional pairs or single supporting arms. In one embodiment, the roller is adapted in this way.

A part or the whole of the roller may be adjustable to change the dimensions of the roller, uses for such a feature may include making it more compact for storage, or increasing the reach of the roller to enable access of different treatment areas. Adjustment may be via a telescopic means, and/or by a means including articulating parts and/or flexible materials. In one embodiment, the roller is adapted in this way.

The roller may be in multiple parts, possibly for the purpose of compact storage, or sterilisation of individual parts.

Any or all of the roller components, and/or the entire roller may be supplied in sterile and/or protective packaging.

The roller may be provided as part of a kit that may comprise any one or more of a sterilising solution, a sanitizing solution, one or more replacement microneedle roller drums which may be fitted to the handle, and one or more arrays of microneedles for fitting to a drum.

The invention may provide a microneedle roller comprising a handle and a drum rotatable relative to the handle, the drum including a plurality of radially projecting microneedles, wherein the handle includes either only one or two arms for supporting the drum, and the roller includes release means for remotely releasing the drum from the said supporting arm(s).

The invention may provide a microneedle roller comprising a rotatable drum, the drum including a plurality of radially projecting microneedles, wherein the roller includes adjustment means for varying the radial projection of at least some of the microneedles relative to the drum.

The invention may provide a microneedle roller comprising a rotatable drum, the drum including a plurality of radially projecting microneedles, further comprising treatment means for treating one or more of the drum, the microneedles, the roller, and the surface over which the roller moves in use.

The microneedle may include any one or more features as described in the first aspect and as claimed herein.

### BRIEF DESCRIPTION OF THE DRAWINGS

The above and other characteristics, features and advantages of the present invention will become apparent from the following detailed description, taken in conjunction with the accompanying drawings, which illustrate, by way of example, the principles of the invention. This description is given for the sake of example only, without limiting the scope of the invention. The reference figures quoted below refer to the attached drawings.

FIG. 1 is a side view of a roller according to one embodiment of the invention;

FIG. 2 is a plan view of the roller of FIG. 1;

FIG. 3 is a plan view of another embodiment of the invention;

FIG. 4 is a side view of yet another embodiment of the invention;

FIG. 5 is a side view of an array of microneedles;

FIG. 6 is a perspective view of a drum for use with the invention; and

FIG. 7 is a perspective cross-sectional view of an alternative drum with microneedle radial projection adjustment means shown.

The present invention will be described with respect to particular embodiments and with reference to certain drawings but the invention is not limited thereto but only by the claims. The drawings described are only schematic and are non-limiting. In the drawings, the size of some of the elements may be exaggerated and not drawn to scale for illustrative purposes. The dimensions and the relative dimensions do not correspond to actual reductions to practice of the invention.

Furthermore, the terms first, second, third and the like in the description and in the claims, are used for distinguishing between similar elements and not necessarily for describing a sequence, either temporally, spatially, in ranking or in any other manner. It is to be understood that the terms so used are interchangeable under appropriate circumstances and that the embodiments of the invention described herein are capable of operation in other sequences than described or illustrated herein.

Moreover, the terms top, bottom, over, under and the like in the description and the claims are used for descriptive purposes and not necessarily for describing relative positions. It is to be understood that the terms so used are interchangeable under appropriate circumstances and that the embodiments of the invention described herein are capable of operation in other orientations than described or illustrated herein.

It is to be noticed that the term “comprising”, used in the claims, should not be interpreted as being restricted to the means listed thereafter; it does not exclude other elements or steps. It is thus to be interpreted as specifying the presence of the stated features, integers, steps or components as referred to, but does not preclude the presence or addition of one or more other features, integers, steps or components, or groups thereof. Thus, the scope of the expression “a device comprising means A and B” should not be limited to devices consisting only of components A and B. It means that with respect to the present invention, the only relevant components of the device are A and B.

Similarly, it is to be noticed that the term “connected”, used in the description, should not be interpreted as being restricted to direct connections only. Thus, the scope of the expression “a device A connected to a device B” should not be limited to devices or systems wherein an output of device A is directly connected to an input of device B. It means that there exists a path between an output of A and an input of B which may be a path including other devices or means. “Connected” may mean that two or more elements are either in direct physical or electrical contact, or that two or more elements are not in direct contact with each other but yet still co-operate or interact with each other.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may refer to different embodiments. Furthermore, the particular features, structures or characteristics may be combined in any suitable manner, as would be apparent to one of ordinary skill in the art from this disclosure, in one or more embodiments.

Similarly it should be appreciated that in the description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the detailed description are hereby expressly incorporated into this detailed description, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those skilled in the art. For example, in the following claims, any of the claimed embodiments can be used in any combination.

In the description provided herein, numerous specific details are set forth. However, it is understood that embodiments of the invention may be practised without these specific details. In other instances, well-known methods, structures and techniques have not been shown in detail in order not to obscure an understanding of this description.

The invention will now be described by a detailed description of several embodiments of the invention. It is clear that other embodiments of the invention can be configured according to the knowledge of persons skilled in the art without departing from the true spirit or technical teaching of the invention, the invention being limited only by the terms of the appended claims.

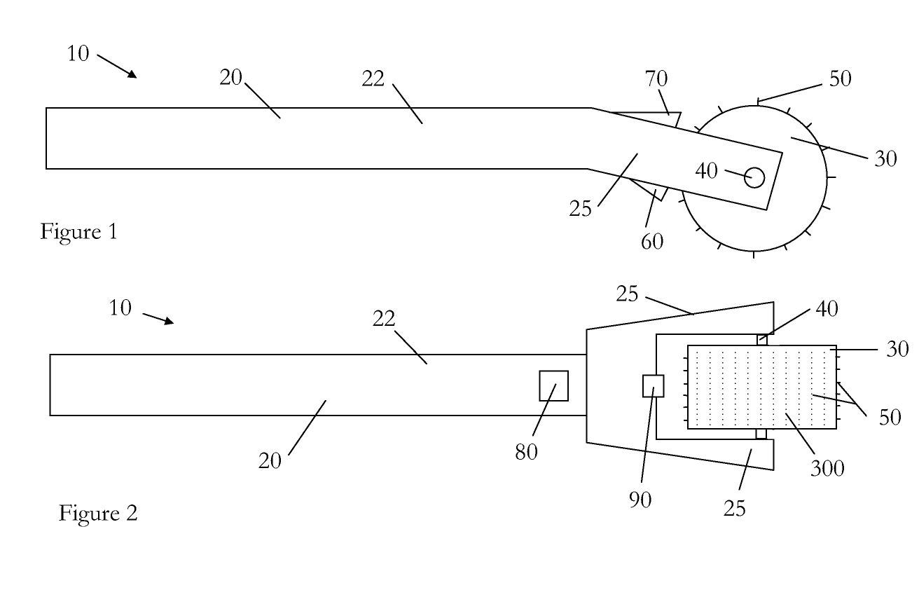

In FIG. 1 a microneedle roller 10 comprises a handle 20. The handle 20 itself comprises two parts; a gripping part 20 and a drum support part 25 at one axial end. The two parts may be unitary or detachably connected together.

A drum 30 is provided which supports a plurality of microneedles 50. The drum is supported by the drum support part 25 by means of an axle 40. The axle 40 allows the drum 30 to rotate relative to the handle 20.

In this and all other relevant figures described herein, the number of microneedles is indicative only and should not necessarily be taken as a true representation of the number or arrangement of microneedles of the invention.

The roller 10 also includes emitters 60, 70. These may emit any one or more of a fluid, an electromagnetic radiation and a sound. The emission may be directed either specifically and/or generally in the direction of the roller 30 and/or the microneedles 50 and/or a surface over which the drum 30 is rolled in use.

FIG. 2 shows the same roller 10 in plan view. The drum support part 25 is bifurcated such that the drum 30 is supported at each end. The roller 10 also includes another emitter 90 located between the forks of the drum support part 25 such that it may direct emissions towards the roller and/or a surface in use.

The handle 20 also includes a button 80. This button may, when manipulated, move the two forks of the drum support part 25 apart such that the drum 30 is released from the roller 10. Alternatively this button may cause the axle or drum to deform allowing release from the roller.

The other referenced features in FIG. 2 are substantially the same as described with regard to FIG. 1.

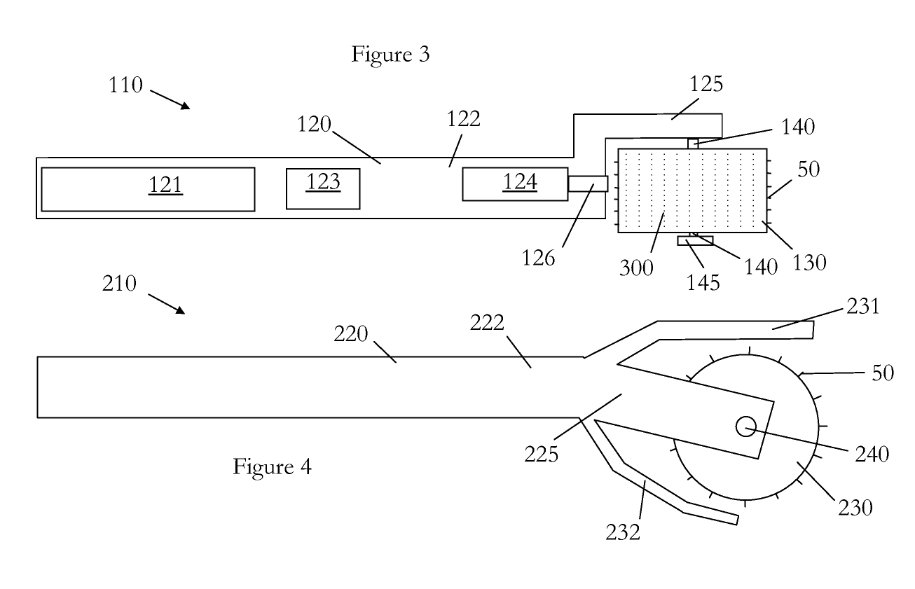

In FIG. 3 an alternative roller 110 is shown. It includes a handle 120 comprising a gripping part 122 and a drum support part 125 in a similar manner to that described with reference to FIGS. 1 and 2.

However, in this embodiment, the drum 130 is only supported at one axial end by the drum support part 125. An axle 140 provides the support between the drum support part 125 and the drum 130. The axle 140 also allows the drum 130 to rotate relative to the handle 120.

The microneedles 50 are present in linear arrays 300 arranged axially across the surface of the drum 130. A button 145 is provided at the opposite axial end of the drum 130 from the drum support part 125. Depression or other manipulation of this button 145 may effect the removal of the drum 130 from the drum support part 125, or adjustment of the radial projection of the microneedles. The handle 120 includes a battery or other power source 121, an ultrasound generator 123 and a light source 124. The light source may emit light via a waveguide 126 onto the drum 130 and/or the microneedles 50 and/or a surface in use. The treatment means 123, 124 although described as an ultrasound generator and a light source may be replaced, or accompanied, by other treatment means such as a fluid source.

FIG. 4 shows another embodiment of the invention. This roller 210 includes a handle 220 comprising a gripping part 222 and a drum support part 225 in a similar manner to that described with reference to FIGS. 1, 2 and 3.

The drum 230 is supported by the drum support part 225. An axle 240 provides the support between the drum support part 225 and the drum 230. The axle 240 also allows the drum 230 to rotate relative to the handle 220.

Furthermore, the roller 210 includes a hood 231 and a comb 232. The hood 231 is positioned substantially radially opposite the comb 232 such that the two partially surround the drum 230 radially. Either, or both, may extend radially around the drum 230 in an arc having a range of 20 degrees to 280 degrees. The hood 231 and/or comb 232 may be integral with, or connected to, the handle 220 and/or the drum support part(s) 225.

Either or both of the hood 231 and the comb 232 may direct treatment means located in the handle 220 towards the drum 230 and/or the microneedles 50 and/or a surface in use.

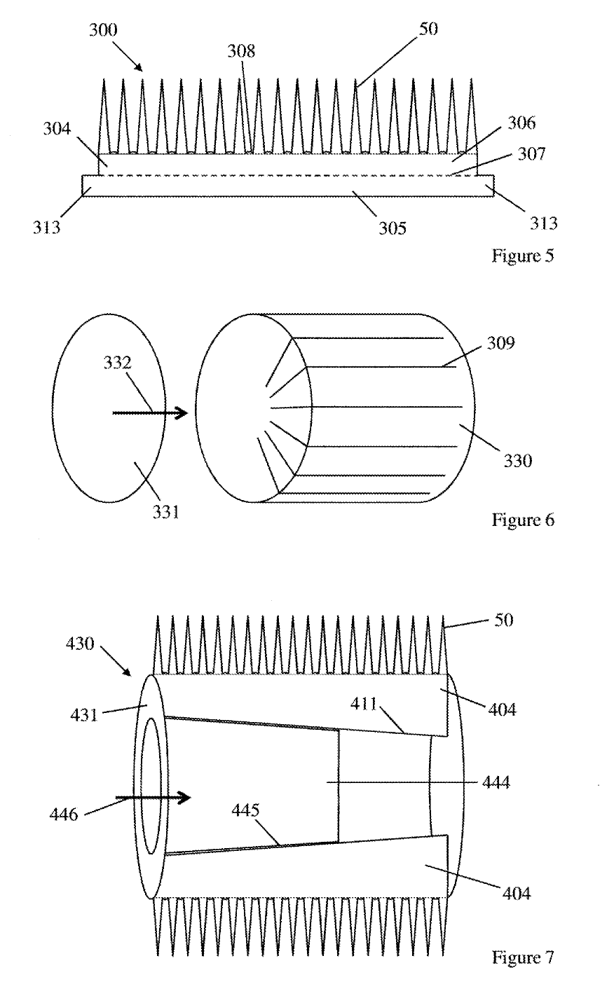

FIG. 5 shows an elevational view of an array 300 of microneedles 50 held by a support 304. The microneedles 50 extend substantially parallel with each other, but in some embodiments the microneedles 50 may diverge or be arranged in a non-parallel manner. Between each microneedle 50 is a roughened surface 308 to provide dermabrasion of a surface in use. The support 304 comprises a base 305 and a microneedle holding part 306. The base 305 and microneedle holding part 306 may be unitary or provided as separate parts, delineated by the broken line referenced 307.

Each end of the array 300 includes a lug 313 for positive engagement with a drum in use.

Such a drum 330 is shown in FIG. 6. The drum 330 includes grooves 309 aligned axially across its outer surface. Although not shown, it is contemplated that the alignment may, in one embodiment, be partially radial and partially axial to produce a spiral-type effect.

An end cap 332 fits over at least one, and possibly both, axial ends of the drum 330 to retain the arrays 300 in place. The end cap 332 is fitted in the direction shown by the arrow referenced 332.

A way of adjusting the radial projection of the microneedles 50 from the outer surface of a drum 430 is shown in FIG. 7. The drum 430 is hollow and includes an adjustment member 444 which is frusto-conical in shape. This member 444 fits through an end cap 431 from one axial end of the drum 430.

Each array 300 includes a support 404 which includes a base having a surface 411 which is inclined relative to the axis of rotation of the drum 430. In use the surface 411 rests against the outer surface 445 of the adjustment member. If the adjustment member 444 is moved in the direction indicated by the arrow referenced 446 then it will be readily apparent that the supports 404 will be pushed radially outward thus increasing the radial projection of the microneedles relative to the outer surface of the drum 430. Conversely, movement of the adjustment member 444 in a direction opposite to direction indicated by the arrow 446 will allow the support 404 to move radially inward thus decreasing the radial projection of the microneedles relative to the outer surface of the drum 430. The movement of the supports 404, and thus the arrays 300, may be aided by resilient means not shown.

What is claimed is:

1. A microneedle roller comprising a handle and a rotatable drum, the drum including a plurality of radially projecting microneedles, the roller further comprising sterilising means in the form of either or both of an ultrasound generator and a UV light for sterilising one or more of the drum, the microneedles, and the surface over which the drum moves in use.

2. The roller of claim 1, wherein the sterilising means comprises at least the ultrasound generator and further comprises a vibration generating means for imparting a vibration to either or both of the roller and the surface in use.

3. The roller of claim 2, wherein the roller is arranged such that the ultrasound generated by the ultrasound generator and the vibration created by the vibration generating means resonate together.

4. The roller of claim 1, wherein the sterilising means comprises at least the ultrasound generator and further comprises a sterilising liquid.

5. The roller of claim 1, wherein the handle comprises a roller hood and/or a comb.

6. The roller of claim 5, being arranged such that, in use, an emission from the sterilising means emanates from the hood and/or comb.

7. The roller of claim 1, being arranged such that, in use, an emission from the sterilising means emanates from the handle and/or the drum.

8. The roller of claim 1, further comprising treatment means, wherein the treatment means has skin and/or hair enhancement properties.

9. The roller of claim 8, wherein the treatment means comprises means to emit one or more of a fluid, a sound, a vibration and an electro-magnetic radiation.

10. The roller of claim 8, wherein the treatment means comprises temperature adjustment means for varying the temperature of either or both of the roller and the surface in use.

11. The roller of claim 8, wherein the treatment means comprises either or both of a brush and a comb.

12. The roller of claim 8, wherein the treatment means comprises either or both of an ultrasound generator and a vibration generating means.

13. The roller of claim 12, wherein the ultrasound generator and vibration generating means are also sterilising means.

14. The roller of claim 8, wherein the treatment means comprises either one of a light and a laser-emitter.

15. The roller of claim 14, wherein the laser-emitter is a cold-beam laser.

16. The roller of claim 14, wherein the light is ultraviolet light.

17. The roller of claim 14, wherein the light is in the visible range of wavelengths.

18. The roller of claim 9, wherein the fluid is one or more of a hair-growth stimulant, a fluid containing a growth factor, a sanitising or sterilising fluid, and a pain or inflammation controlling fluid.

19. The roller of claim 1, further comprising abrasion means for abrading a surface over which it moves in use.

20. A kit including the roller of claim 1 and any one or more of a sterilising solution, a sanitizing solution, one or more replacement microneedle roller drums which may be fitted to the handle, and one or more arrays of microneedles for fitting to a drum.

21. The roller of claim 9, wherein the treatment means comprises temperature adjustment means for varying the temperature of either or both of the roller and the surface in use.
